(19) United States
(12) Patent Application Publication
(10) Pub. No.: US 2023/0270762 A1
(43) Pub. Date: Aug. 31, 2023

(54) TOPICAL COMPOSITION FOR THE TREATMENT OF PSORIASIS AND RELATED SKIN DISORDERS

(71) Applicant: BIODERM, INC., Madison, WI (US)

(72) Inventor: Lorraine Faxon Meisner, Madison, WI (US)

(21) Appl. No.: 18/005,507

(22) PCT Filed: Feb. 19, 2021
(86) PCT No.: PCT/US2021/018835

§ 371 Date: Jan. 13, 2023

Related U.S. Application Data
(60) Provisional application No. 63/053,103, filed on Jul. 17, 2020.

(51) Int. Cl. A61K 31/00; A61K 31/4375; A61K 31/7008; A61K 31/7048; A61K 31/60; A61K 35/04; A61K 9/00; A61P 17/06

(52) U.S. Cl. CPC A61K 31/658; A61K 31/4375; A61K 31/7008; A61K 31/7048; A61K 31/60; A61K 35/04; A61K 9/0014; A61P 17/06

(57) ABSTRACT

The present disclosure provides compositions and methods of use thereof for the treatment of skin diseases and disorders. The compositions comprise cannabidiol, or derivatives or pharmaceutical salts thereof. The formulations may also include glucosamine and antioxidant anti-inflammatory herbal extracts such as oleuropein and berberine in an emollient base.

### CROSS-REFERENCE TO RELATED APPLICATIONS

This application claims the benefit of U.S. Provisional Application No. 63/053,103, filed Jul. 17, 2020, the contents of which is incorporated herein by reference.

### FIELD

The present disclosure provides novel compositions and methods for treatment of psoriasis and related inflammatory skin disorders. More particularly the current invention pertains to a composition comprising cannabidiol (CBD) or derivatives thereof.

### BACKGROUND

Without limiting the scope of the invention, its background is described in connection with disorders of the skin and, more particularly, to the general field of diseases that cause psoriasis, as an example.

Psoriasis is a common noncontagious and chronic inflammatory autoimmune skin disease characterized by hyperplasia of keratinocytes resulting in thickening of the epidermis and the presence of red scaly plaques. The lesions in this chronic disease typically are subject to remissions and exacerbations. There are several patterns, of which plaque psoriasis is the most common. Guttate psoriasis, with raindrop shaped lesions scattered on the trunk and limbs, is the most frequent form in children, while pustular psoriasis is usually localized to the palms and soles. The classical inflammatory lesions vary from discrete erythematous papules and plaques covered with silvery scales, to scaly itching patches that bleed when the scales are removed. Symptoms of psoriasis can fluctuate and at intermittent intervals may spontaneously get better or worse. While psoriasis is not curable, it can go into remission at which times the skin appears normal and is clear. People with psoriasis have a higher risk developing lifelong co-morbidities, especially when left untreated, which includes hypertension, hyperlipidemia, cardiovascular disease, inflammatory bowel disease, liver problems, psoriatic arthritis, obesity, metabolic syndrome, and depression. Over time, these untreated psoriasis skin changes can cause unnecessary stress to the affected individual and can lead to other health problems. Psoriasis can lead to anxiety and depression, as individuals with the skin disorder are often afraid of being ridiculed.

Atopic Dermatitis (AD) is one of the most prevalent skin diseases, affecting up to 20 percent of the population in the developed world and with a predilection for children. It affects 15-25% of children, and almost one third of the cases persist into adulthood. AD has a significant disease burden, both taxing economically and decreasing the quality of life for patients. AD or atopic eczema is a common chronic inflammatory disorder of the skin and is one of the most prevalent skin diseases, that like psoriasis has an auto immune component. AD is characterized by mutations in genes affecting the barrier function of the stratum corneum, leading to increased epidermal water loss as well increased risk of microbial invasion. Past treatments for AD involve emollients, topical corticosteroids, topical tacrolimus, topical calcineurin inhibitors, and in resistant cases, ultra-violet radiation.

Despite a voluminous scientific literature and numerous treatment strategies, there is still no effective treatment for psoriasis or atopic dermatitis that is completely without side effects. Conventional therapies to treat psoriasis range from the use of biologics, oral treatments, phototherapy, and topical applications to lifestyle choices such as a change in diet or introduction of vitamins and supplements. Usually treated with Humira and Enbrel, psoriasis comes with a shocking price tag and complications that are often difficult to ignore. The number of different and sometimes toxic treatments employed for amelioration of psoriasis is testimony to the resistant nature of this disease. Not only is moderate to severe psoriasis resistant to topical treatments, but because of its chronic and recurrent nature, systemic therapy or radiation is often required. The devastating nature of this disease is emphasized by the extent of the side effects that psoriasis sufferers are willing to endure to attain a remission to a disease that they know will recur sooner or later.

### SUMMARY

Disclosed herein are compositions comprising: at least 0.1% by weight CBD, or a pharmaceutically acceptable salt thereof; at least 5% by weight glucosamine, or a pharmaceutically acceptable salt thereof; at least 1% by weight berberine, or a pharmaceutically acceptable salt thereof; and at least 0.5% by weight oleuropein, or a pharmaceutically acceptable salt thereof.

Also disclosed herein are methods of treating skin diseases or disorders comprising topically applying a therapeutically effective amount of the composition described herein. In some embodiments, the skin disease or disorder may comprise psoriasis, eczema, or atopic dermatitis.

### BRIEF DESCRIPTION OF THE DRAWINGS

FIGS. 1A and 1B are before and after images, respectively, of treatment of psoriasis with an exemplary composition of the present disclosure.

FIGS. 2A and 2B are before and after images, respectively, of treatment of eczema with an exemplary composition of the present disclosure.

### DETAILED DESCRIPTION

Before any embodiments of the methods and compositions of the present disclosure are explained in detail, it is to be understood that the inventions disclosed herein are not limited in application to the details set forth in the description or illustrated in the drawings. The inventions are capable of other embodiments and of being practiced or of being carried out in various ways.

The present invention employs an emollient base such as moisturizing agents to promote skin re-epithelialization thereby diminishing disfiguring lesions. In addition, the present invention also addresses the underlying T-cell disorder that results in an inflammatory condition. Most, if not all, of the current therapies for psoriasis or similar T-cell mediated inflammatory skin conditions are designed to kill T-cells and to thereby ameliorate inflammation. It is possible that a major problem with the current treatments is that the therapy itself is so toxic that it may promote recurrence during healing. The toxicity of current treatments unleashes some or all the cytokines that are associated with the promulgation of these chronic and often rebounding skin diseases.

Chronic inflammation leads to hyperproliferation and angiogenesis, and agents that control inflammation also control angiogenesis and hyperproliferation. A prime example is corticosteroids, which are generally effective for treatment of psoriasis as well as atopic dermatitis. Corticosteroids' side effects, however, include decreased connective tissue synthesis, weakened blood vessels due to the diminished connective tissue support, bone loss, increased infection, etc. When corticosteroids are discontinued, the disease returns more aggressively and more resistant to therapy. As such, biologics are increasingly used to treat psoriasis as well as atopic dermatitis.

It is because of the harsh treatment strategies that dominate psoriasis therapy that the following formulations were designed, the objective of which is to preserve the skin surfaces without subjecting the cells to hyperproliferation, without compromising the immune system, and without causing changes that result in an increased risk of skin cancer or other conditions. In the present invention the composition selected is capable of topical administration to have a localized effect, completely non-toxic to normal skin, and an anti-inflammatory agent. The composition comprises CBD and glucosamine in an emollient base and is shown herein to be an effective therapy for psoriasis and related skin ailments. D-glucosamine HCL was selected as one of the agents in the topical formulation for treatment of inflammatory skin diseases which, unlike the situation in wound healing, must work to oppose the activity of the T-cells. Such a formulation may include CBD, glucosamine and extract from at least one herb that elicits at least one of the following biological effects: anti-inflammatory, antioxidant, antibacterial, antimicrobial, anti-pruritic, anti-platelet adhesion, vasodilation or keratolysis. A number of appropriate herbs that may work, for the present invention, are known to those of skill in the art in light of the present disclosure. Two herbs were selected that appear to aid in the treatment of skin conditions are oleuropein and berberine. The components in the composition are non-irritating and able to be absorbed topically making them particularly suited for treatment of compromised skin.

The composition also has anti-itch functionality important for use in treating pruritus caused by psoriasis and eczema without corticosteroids and also for eliminating itch from insect bites, poison ivy and environmental pollutants. In addition, the composition may decrease swelling as a result of insect bites, poison ivy and irritation from environmental pollutants.

The present invention also includes methods for the treatment of skin diseases or disorders. The method comprises topically applying the composition as disclose herein to the affected skin to treat and ease the pain and discomfort caused by skin diseases and disorders. The composition provides a non-toxic highly effective treatment for psoriasis and other skin conditions without the side effects observed with virtually all other therapies for moderate to severe psoriasis (mild psoriasis may be successfully treated with proper moisturizing) and other skin conditions.

Section headings as used throughout the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can.” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,”-consisting of and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example. “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “cannabidiol (CBD)” refers hereinafter to one of at least 85 active cannabinoids identified in Cannabis sativa. It is a 21-carbon terpenophenolic compound which is formed following decarboxylation from a cannabidiolic acid precursor. CBD can be isolated from cannabis or be produced synthetically. Cannabidiol is a major phytocannabinoid, accounting for up to 40% of the plant's extract. Cannabidiol has a very low affinity for CB1 and CB2 receptors but acts as an indirect antagonist of their agonists. CBD may potentiate THC's effects by increasing CB1 receptor density or through another CB1-related mechanism. It is also an inverse agonist of CB2 receptors. CBD possesses antiproliferative, pro-apoptotic effects and inhibits cancer cell migration, adhesion and invasion.

The term “tetrahydrocannabinol (THC)” refers hereinafter to the principal psychoactive constituent (or cannabinoid) of the cannabis plant, although a minor component of C. sativa. THC has a partial agonist activity at the cannabinoid receptor CB1, and the CB2 receptor.

The term “cannabinoid receptor” refers hereinafter to a class of cell membrane receptors under the G protein-coupled receptor superfamily. There are currently two known subtypes of cannabinoid receptors, termed CB1 and CB2. The CB1 receptor is expressed mainly in the central and peripheral nervous system, but also in the lungs, liver and kidneys. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC. The CB2 receptor is expressed mainly in the immune system and in hematopoietic cells. It is closely related to the cannabinoid receptor type 1, which is largely responsible for the efficacy of endocannabinoid-mediated presynaptic-inhibition, the psychoactive properties of Tetrahydrocannabinol, the active agent in marijuana, and other phytocannabinoids (natural cannabinoids). The principal endogenous ligand for the CB2 receptor is 2-arachidonoylglycerol (2-AG).

The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids.

The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment).

As used herein, “treat,” “treating” and the like means a slowing, stopping or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

### 2. COMPOSITIONS

Disclosed herein is a composition comprising: at least 0.1% by weight CBD, or a pharmaceutically acceptable salt thereof; at least 5% by weight glucosamine, or a pharmaceutically acceptable salt thereof; at least 1% by weight berberine, or a pharmaceutically acceptable salt thereof; and at least 0.5% by weight oleuropein, or a pharmaceutically acceptable salt thereof.

The composition may comprise at least 0.1% by weight CBD, or a pharmaceutically acceptable salt thereof. In some embodiments, the composition comprises between about 0.1-10% CBD by weight. In certain embodiments, the composition comprises between about 0.1-1% by weight. In exemplary embodiments, the composition comprises between 0.2% and 0.5% by weight CBD.

The composition may comprise at least 5% by weight glucosamine, or a pharmaceutically acceptable salt thereof. In some embodiments, the composition comprises between about 5-25% glucosamine by weight.

Although the mechanism of action of glucosamine is not well understood, it was shown that, in vitro, it significantly increases secretion of mucopolysaccharides by fibroblasts. This contrasts with the effects of steroids and non-steroidal anti-inflammatory drugs, which inhibit mucopolysaccharide metabolism by fibroblasts in vitro, and appear to decrease connective tissue in vivo. Thus glucosamine, though anti-inflammatory, does not compromise normal connective tissue as do other anti-inflammatory agents.

Without being bound by theory, glucosamine may work by inhibiting T-cell access to the skin as a result of the increased density of the connective tissue promoted by glucosamine. In contrast, following the use of the other agents the connective tissue tends to be compromised, leaving the skin more accessible and vulnerable to cellular infiltration. Therefore, the effect of glucosamine on T-cell induced inflammation may be explained due to the relationship of dense skin and attenuation of cytokines. Denser skin, with increased mucopolysaccharides promoted by glucosamine, may attenuate the cytokines elaborated by the activated T-cells. Attenuation of the T-cell cytokines may then inhibit the inflammatory effect of the cytokines, possibly through dilution. Even in dense young skin, this may be the effect of the glucosamine: to bind nonspecifically to cytokines or to entrap the cytokines in a mucopolysaccharide “net”, thereby inhibiting the inflammatory effect of the cytokines on the skin.

It has been suggested that oral glucosamine might be effective in treating psoriasis, but experiments demonstrate that topical application of glucosamine is preferred for a primary effect on the skin. The effect of glucosamine on arthritis suggests that it may be a systemic anti-inflammatory agent, but systemic anti-inflammation may not be desirable or preferred for the treatment of psoriasis and related skin ailments.

The compositions may comprise at least 1% by weight berberine, or a pharmaceutically acceptable salt thereof. In some embodiments, the composition comprises between about 1-10% by weight berberine.

The berberine may be from an herbal source. In some embodiments, the berberine is a component of a grape seed extract.

Berberine is also said to possess antimicrobial activity, as well as being antifibrotic, anti-platelet adhesion and a natural protectant against heart disease and circulatory complaints. It has been widely used for the treatment of inflammation in Chinese herbal medicines and has also been used to treat diarrhea in dysentery, as well as to treat non-insulin dependent diabetes mellitus. Without going exhaustively into the list of conditions for which berberine has shown a possible effect, which includes those listed above as well as for chloroquine resistant malaria and for treating ventricular tachyarrhythmias by improving left ventricular function with the production of mild systemic vasodilation; it is clear that this compound has a long track record for safety.

The composition may comprise at least 0.5% by weight oleuropein, or a pharmaceutically acceptable salt thereof. In some embodiments, the composition comprises between about 0.5-7.5% by weight oleuropein.

The oleuropein may be from an herbal source. In some embodiments, the oleuropein is a component of an olive leaf extract.

Oleuropein is a glucoside to which a great many properties have been attributed in the herbal literature. It may work in the present formulation by helping to restore the normal health of the skin by aiding in repair. Oleuropein has been called a natural antibiotic because it has been claimed to relieve symptoms of all types of infections: fungal, bacterial, viral, and parasitic. Any agent to which so many different mechanisms are attributed is generally suspect, but it has been suggested that there is an anti-viral constituent in oleuropein, calcium enolate, which is obtained after mild acid hydrolysis, and is said to work by inactivating viruses by dissolving their outer envelope. Aside from claims in the health food industry, it has long been known in Greece that during the olive harvest, the skin problems of those climbing the trees improved considerably.

The composition may comprise a ratio of glucosamine to berberine to oleuropein of approximately 9:1.75:1, respectively.

The composition may further comprise a keratolytic agent. Keratolytic agents include those which improve the skin's moisture binding capacity including, but not limited to, alkalis, salicylates, such as salicylic acid, urea, lactic acid, allantoin, glycolic acid, and trichloroacetic acid. In some embodiments, the keratolytic agent comprises coal tar extract. In some embodiments, the keratolytic agent comprises a salicylate. The salicylate may comprise salicylic acid.

The composition may further comprise an emollient. The emollient base may include a large spectrum of suitable substances, including but not limited to creams, moisturizing creams, ointments, oils, waxes, gels, lotions, liquid suspensions or dispersions, emulsions, emulsions comprising oil in water, and the like, provided the emollient base is suitable for topical application on the skin, is substantially non-toxic and provides a suitable carrier for the non-emollient medicinal agents of the invention. A properly chosen emollient base may provide a certain amount of relief for mild outbreaks of psoriasis or dermatitis. In some embodiments, the emollient comprises a moisturizing cream.

The compositions may be formulated for administration by, for example, topical formulations. Techniques and formulations may generally be found in “Remington's Pharmaceutical Sciences,” (Meade Publishing Co., Easton, Pa.). Therapeutic compositions must typically be sterile and stable under the conditions of manufacture and storage.

The disclosed compositions can be topically administered. Topical compositions that can be applied locally to the skin may be in any form including solids, solutions, oils, creams, ointments, gels, lotions, foundations, shampoos, leave-on and rinse-out hair conditioners, milks, cleansers, moisturizers, sprays, skin patches, and the like. Topical compositions include: a disclosed composition and a carrier. The carrier of the topical composition preferably aids penetration of the ingredients. The carrier may further include one or more optional components. The amount of the carrier employed in conjunction with a disclosed composition is sufficient to provide a practical quantity of composition for administration per unit dose.

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

Carriers for topical application which may be used with the present invention include, but are not limited to, alkyleneglycols, or alkyleneglycols in combination with one or more derivatives of hydroxyalkylcellulose. In one illustrative embodiment, the alkylene glycol is propyleneglycol and the hydroxyalkylcellulose is hydroxypropylcellulose.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethyl sulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

The composition may be formulated for use on humans or non-human animals (e.g. companion animals). For example, dogs, like humans, are at risk for similar skin diseases such as psoriasis, pruritis, atopic dermatitis, allergies and irritation (among others). A major cause of skin irritation in dogs is related to pesticides and herbicides frequently used on lawns, fleas, ticks and other bugs and insects, flowers, shrubbery and plants that make them vulnerable to allergies and irritation. Since, dogs are frequently exposed to these environment the results cause problematic skin issues, some of which can be serious. Moreover, horses, the only species other than primates with sweat glands also suffer from human skin disorders, such as psoriasis and eczema. In some embodiments, the composition is a veterinary cream, lotion, shampoo, or the like.

### 3. METHODS OF TREATMENT

Disclosed herein is a method of treating skin disease or disorder comprising topically applying a therapeutically effective amount of the composition described herein to the affected skin. The skin disease or disorder may comprise psoriasis, eczema, or atopic dermatitis. The application can be applied as needed to control the condition, for example, when severe itching, redness or scabs develop. For example, the compositions can be applied once daily, twice daily, every four hours, etc. for the duration of the flare ups or symptoms.

Psoriasis lesions may be disfiguring and often result in psychological problems. Numerous psoriasis support groups exist to help suffers cope with the disease. It is generally a lifelong disease with exacerbations and remissions, with a mean age of onset of 27.8 years. Two percent of cases occur in infants. Psoriasis is estimated to affect two percent of the U.S. population, and its worldwide prevalence is 0.1 to 3 percent. There is a significant genetic component, since 35 percent of patients have at least one affected relative. The lifetime risk without affected relatives is 4 percent, but it is 28 percent if one parent is affected, and 65 percent if both parents are affected.

Under the broader diagnosis of psoriasis, there are 5 different types of skin conditions. Subjects with psoriasis may have more than one type or different types of psoriasis at different times throughout their lifetime. The composition may be used with any of the types of psoriasis.

Although psoriasis manifests as a skin disorder, it is believed to be a disease of impaired or defective cell-mediated immunity. Dysregulated lymphocytes produce cytokines that stimulate the proliferation of apoptosis-resistant keratinocytes. The same resistance to therapy also characterizes atopic dermatitis, another skin condition associated with T-cell activation with elaboration of cytokines that lead to epidermal hyperplasia. Atopic dermatitis also has a genetic component, though it is less well defined than for psoriasis. Atopic dermatitis is also characterized by disfiguring red scaly lesions, inflammation, and resistance to therapies that are almost the same as those used to treat psoriasis.

The compositions disclosed herein may be administered alone or in combination with a therapeutically effective amount of at least one additional therapeutic agents or therapeutic regimen. The additional therapeutic agent(s) or regimens may be administered simultaneously or sequentially with the disclosed compositions. Sequential administration includes administration before or after the disclosed compositions. In some embodiments, the additional therapeutic agent or agents may be administered in the same composition. In other embodiments, there may be an interval of time between administration of the additional therapeutic agent and the disclosed composition. In some embodiments, administration of an additional therapeutic agent with a disclosed composition may allow lower doses of the other therapeutic agents and/or administration at less frequent intervals. When used in combination with one or more other active ingredients, the compositions of the present invention and the other active ingredients may be used in lower doses than when each is used singly.

Topical treatments that may be used in combination with the compositions disclosed herein may include other topical therapies. Topical therapies may include coal tar preparation (1-5% by weight). Although this is the most frequently used topical therapy, coal tar has a bad odor and stains clothing. Coal tar is thought to be effective for psoriasis because it is toxic to T cells but is not toxic to skin cells.

Another mainstay of topical therapy includes topical steroids, but long-term use of fluorinated corticosteroids (which are more effective than hydrocortisone) may lead to striae, telangiectasis and ecchythmosis. Topical anthralin cream (1%) or high dose/short duration anthralin in 1% salicylic acid in petroleum may be effective, or the topical synthetic retinoid tazarotene, may also provide short-term relief, although these are often irritating. Other treatments involving retinoids are described, for example, in U.S. Pat. Nos. 3,934,028; 3,966,967; 4,021,573 and 4,216,224. Other topical agents such as calcipotriene, a vitamin D analogue (vitamin D3 or calcipotriol) may also provide temporary relief, while keratolytics such as salicylic acid can help in removing the thick scales from the psoriatic plaques. See, for example, U.S. Pat. No. 4,483,845. Coal tar extract, the most common topical treatment, is generally considered to be a mild skin irritant, a weak antiseptic and a keratolytic agent. Nevertheless, coal tar extract and salicylates are generally considered substantially non-toxic in spite of a certain amount of skin irritation associated with their use. Despite their limited effectiveness and the discomfort associated with their use, these topical therapies are the mainstay of treatment for moderate psoriasis while they may be used as adjunctive therapy in patients with more severe disease.

It is commonly observed that natural sunlight may be beneficial for treatment of psoriasis, and this has led to the use of UV radiation therapy. Examples are shown in U.S. Pat. Nos. 4,153,572 and 4,558,700. UVB radiation (280-320 nm) of affected areas is one of the most common treatments for moderately severe psoriasis, with its efficacy enhanced by coating the skin with a tar containing emollient prior to the radiation. UVA radiation, with longer wavelength (320-380 nm), enables the radiation to extend into the dermal layer. One common form of treatment requires that the patients ingest psoralen orally, and receive UVA radiation about an hour later, which is why this treatment is called PUVA.

Other systemic therapy includes X-ray to the affected regions, as well as oral corticosteroids, because of its immunosuppressive effect on the cytotoxic T lymphocytes. Another frequently used chemotherapeutic agent is methotrexate, which is particularly beneficial in patients with psoriasis, although its disadvantages include leukopenia and cumulative hepatic toxicity that requires frequent monitoring by fine needle biopsies of the liver. Cyclosporine has been approved for treatment of severe psoriasis, although long term therapy with this drug may result in hypertension and potential nephrotoxicity. Hydroxyurea, another cancer chemotherapeutic agent, is moderately effective in controlling psoriasis, but its use is also limited by hematologic side effects. Acitretin, a synthetic retinoid, may also be beneficial although retinoids are teratogenic, and patients using acitretin may experience extreme dryness of mucous membranes and an increase in arthralgias, as well as increased blood triglycerides and, less commonly, increased blood levels of cholesterol and hepatic enzymes may occur.

In addition to the therapies discussed above, the following United States Patents are examples of other modalities that have been developed for psoriasis, illustrating the sometimes-extreme measures some people will take to treat the disease: U.S. Pat. Nos. 4,788,057, 4,853,388, 5,501,705, 5,527,350, 5,760,006, 5,800,831, 5,833,996, 5,836,999, and 5,976,505

Similarly, effective therapeutic agents are also limited for treatment of atopic dermatitis, and even effective therapies often have a very short-term effect. Steroids are currently the most widely used topical treatment, but since these are often ineffective in controlling inflammation, UVA or UVB therapy is also used; as is coal tar.

Immunosuppressive therapy using cyclosporine, topical tacrolimus (used in organ transplantation); methotrexate (though it is not as effective as it is in psoriasis), and other similar chemotherapeutic agents are also used because of their effects on T-cell mediated immune responses, as are azathioprene and interferon gamma. Yet, although these aggressive therapies may produce remissions, the remissions tend to be brief and often soon require additional therapy, subjecting the patient to the choice between toxic therapies and a devastating skin disease.

The composition should be suitable for topical application on human skin and may at least partially suppress, local to the area of topical application, the production of at least one cytokine that stimulates the proliferation of apoptosis-resistant keratinocytes.

The compositions may be used for humans or non-human animals (e.g. companion animals). In animals, the compositions may be additionally be useful for treatment of reactions to fleas, parasites, or other non-human infectious diseases. The disclosed compositions treat the symptomatic causations of pet itching without damaging the sensitive coat or skin of animals or causing other adverse side effects. In some embodiments, the composition is used on non-human animals to treat a flea allergy, pruritus, equine skin disease, or seborrhea.

### 4. EXAMPLES

### Example 1

### Treatment of Severe Chronic Skin Diseases

Composition as disclosed herein comprising 0.5% CBD and associated hemp oil, glucosamine and plant extracts are used to treat moderate to severe plaque psoriasis including atopic dermatitis. Controls consist of each of the active ingredients and 1-2% salicylic acid. Level and amount of irritation, number of days until improvement, level and number of psoriatic plaques, length of time until relapse are monitored over 60 days with periodic follow-up for recurrence data points. The Psoriasis Area and Severity Index is used to measure the severity of psoriasis and combines the assessment of the severity of lesions with the area affected into a single score.

### Example 2

### Treatment of Psoriasis and Eczema

Compositions as disclosed herein were applied twice daily to affected areas and at least one half of an inch of the adjacent normal skin in patients diagnosed with psoriasis, eczema, atopic dermatitis, and/or another skin disease.

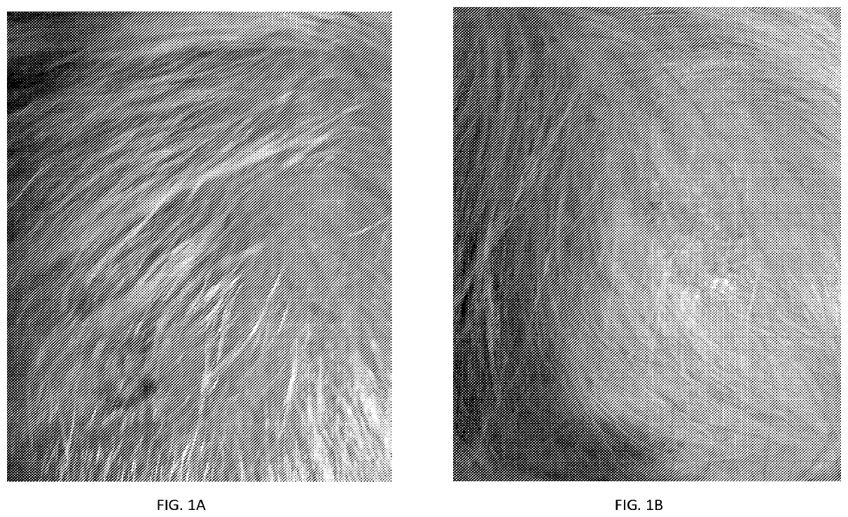

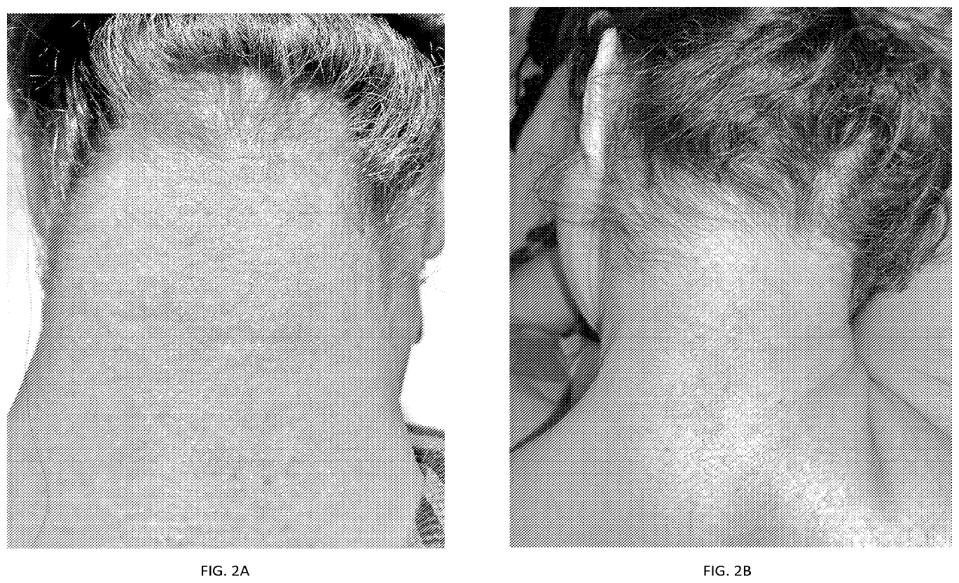

FIGS. 1A and 1B show before and after images, respectively of treatment of psoriasis on the head of a patient. FIGS. 2A and 2B show before and after images, respectively of treatment of eczema on the neck area of a patient. Overall, patients reported experiencing less itching, clearance of scabs, and lessening of redness in affected areas after 2-4 weeks of treatment.

It is understood that the foregoing detailed description are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. A composition comprising:
at least 0.1% by weight cannabidiol (CBD), or a pharmaceutically acceptable salt thereof;
at least 5% by weight glucosamine, or a pharmaceutically acceptable salt thereof;
at least 1% by weight berberine, or a pharmaceutically acceptable salt thereof; and
at least 0.5% by weight oleuropein, or a pharmaceutically acceptable salt thereof.

2. The composition of claim 1, wherein glucosamine comprises between about 5-25% of the composition by weight.

3. The composition of claim 1 or claim 2, wherein berberine comprises between about 1-10% by weight of the composition.

4. The composition of any of claims 1-3, wherein oleuropein comprises between about 0.5-7.5% by weight of the composition.

5. The composition of any of claims 1-4, wherein the CBD comprises between about 0.1-10% by weight of the composition.

6. The composition of any of claims 1-5, wherein the CBD comprises between about 0.1-1% by weight of the composition.

7. The composition of any of claims 1-6, wherein the berberine is from an herbal source.

8. The composition of any of claims 1-7, wherein the berberine is a component of a grape seed extract.

9. The composition of any of claims 1-8, wherein the oleuropein is from an herbal source.

10. The composition of any of claims 1-9, wherein the oleuropein is a component of an olive leaf extract.

11. The composition of any of claims 1-10, further comprising a keratolytic agent.

12. The composition of any of claims 1-11, wherein the keratolytic agent comprises coal tar extract.

13. The composition of any of claims 1-12, wherein the keratolytic agent comprises a salicylate.

14. The composition of claim 13, wherein the salicylate comprises salicylic acid.

15. The composition of any of claims 1-14, further comprising an emollient.

16. The composition of claim 15, wherein the emollient comprises a moisturizing cream.

17. A composition of claim 1, wherein the relative amounts of glucosamine, berberine and oleuropein comprise a ratio of approximately 9:1.75:1 of glucosamine, berberine and oleuropein, respectively.

18. A method of treating a skin disease or disorder in a subject comprising topically applying a therapeutically effective amount of the composition of any one of claims 1-17 to the affected skin.

19. The method of claim 18, wherein the skin disease or disorder comprises psoriasis, eczema or atopic dermatitis.

20. The method of claim 18, wherein the skin disease or disorder comprises inflammation, rashes, allergic reactions, pain, or combinations thereof.

21. The method of claim any of claims 18-20, wherein the subject is a human or non-human mammal.

22. The method of claim 21, wherein the subject is a dog or horse.

23. The method of claim 22, wherein the disease or disorder comprises a flea allergy, pruritus, equine skin disease, or seborrhea.
